(19) United States
(12) Patent Application Publication
(10) Pub. No.: US 2025/0144294 A1
(43) Pub. Date: May 8, 2025

(54) IMPLANTABLE DEVICE FOR AUTOMATIC DRUG ADMINISTRATION

(71) Applicant: SEOUL NATIONAL UNIVERSITY R&DB FOUNDATION, Seoul (KR)

(72) Inventors: Young Bin CHOY, Seoul (KR); Cho Rim KIM, Seoul (KR); Min Ji KIM, Anyang-si (KR)

(21) Appl. No.: 19/016,014

(22) Filed: Jan. 10, 2025

Related U.S. Application Data
(63) Continuation of application No. PCT/KR2023/009827, filed on Jul. 11, 2023.

(30) Foreign Application Priority Data
Jul. 15, 2022 (KR) 10-2022-0087244

(51) Int. Cl. A61M 5/172; A61M 5/20

(52) U.S. Cl. CPC A61M 5/172; A61M 5/2033; A61M 2005/202; A61M 2205/8206

(57) ABSTRACT

An implantable device for automatic drug administration includes a housing; a drug release unit configured to eject a drug; and a driving unit configured to drive the drug release unit at preset intervals, wherein the drug release unit comprises a drug storage, a drug chamber receiving a drug from the drug storage, and a drug release controller configured to fill the drug chamber with a drug received and release the drug filled in the drug chamber. The drug release controller comprises a plunger configured to form one of positive pressure and negative pressure in the drug chamber as the plunger moves, a plunger spring configured to apply a restoring force to a position where the plunger applies presses the drug chamber, and a gear portion connected to the driving unit and configured to rotate at a preset time to drive the drug release unit.

### CROSS-REFERENCE TO RELATED APPLICATION

This application is a continuation of International Application No. PCT/KR2023/009827, filed on Jul. 11, 2023, which is based on and claims priority to Korean Application No. 10-2022-0087244, filed on Jul. 15, 2022, in the Korean Intellectual Property Office, the entire disclosures of which are incorporated herein by reference.

### BACKGROUND

### 1. Field

The present disclosure relates to an implantable device for automatic drug administration.

### 2. Description of the Related Art

Chronic diseases usually last for more than three months, such as diabetes, osteoporosis, cardiovascular disease, and growth hormone deficiency and cause pain to patients regardless of age, rank, or race. Long-term medication treatment methods are commonly used to treat such chronic diseases and include oral administration, injection treatment, and implantable devices.

However, oral administration has the disadvantage that the drug activity decreases or is denatured as it passes through the gastrointestinal tract, which reduces bioavailability and has the limitation that long-term administration may cause gastrointestinal side effects.

Also, in the case of the syringe injection, frequent syringe injection over a long period of time causes pain to patients, and causes inconveniences such as a cost burden and frequent hospital visits by patients. In order to solve the problem, the development of implantable devices that may efficiently deliver drugs for a long period of time with a single implant is in progress, but in most cases, the implantable devices have the disadvantage of being difficult to use permanently and miniaturize because each of the implantable devices include batteries, electronic circuits, and so on.

Also, in the case of on-demand implantable devices that release drugs through external stimulation when drug administration is required, there is a difficulty in that patients have to remember the daily dosing time and administer drugs themselves, which has the limitation of lowering drug compliance in patients who take drugs every day.

Therefore, the development of an implantable drug delivery device that may increase the convenience and treatment effectiveness of patients with chronic diseases is required.

### PRIOR ART

Korea Patent Publication No. 10-2021-0096382 (Invention Title: Implantable and Manually-controllable Drug Delivery Device)

### SUMMARY

One embodiment of the present disclosure provides an implantable device that may be miniaturized and has an automatic drug administration function for automatically administering drugs after implantation in a patient's body.

However, technical tasks that the present embodiment seeks to accomplish are not limited to the technical tasks described above, and there may be other technical tasks.

According to an aspect of the present disclosure, an implantable device for automatic drug administration includes a housing having a predetermined shape; a drug release unit configured to eject a drug to an outside; and a driving unit configured to drive the drug release unit at preset intervals, wherein the drug release unit includes a drug storage located inside the housing and storing a drug, a drug chamber receiving a predetermined amount of drug from the drug storage and storing the received drug, and a drug release controller configured to fill the drug chamber with a drug received the drug storage and release the drug filled in the drug chamber to the outside. In this case, the drug release controller includes a plunger configured to form one of positive pressure and negative pressure in the drug chamber as the plunger moves, a plunger spring configured to apply a restoring force to a position where the plunger applies presses the drug chamber, and a gear portion connected to the driving unit and configured to rotate at a preset time to drive the drug release unit.

According to the present disclosure, there is an effect of providing an implantable device having an automatic drug administration function that may be miniaturized with a simple mechanical configuration and automatically administer drugs after implantation into a patient's body.

### BRIEF DESCRIPTION OF THE DRAWINGS

FIG. 1 is a perspective view of an implantable device for automatic drug administration according to an embodiment of the present disclosure.

FIG. 2 is a schematic view of a driving unit according to a first embodiment of the present disclosure.

FIG. 3A to FIG. 3C are views illustrating an operating mechanism of a driving unit according to the first embodiment of the present disclosure.

FIG. 4 is a schematic view of a driving unit according to a second embodiment of the present disclosure.

FIG. 5A and FIG. 5B are views illustrating operating mechanisms of a driving unit according to the second embodiment of the present disclosure.

FIG. 6A to FIG. 6C are views illustrating a drug administration time controller according to an embodiment of the present disclosure.

FIG. 7 is a schematic view of a driving unit according to an embodiment of the present disclosure.

FIG. 8A and FIG. 8B are views illustrating a manual charging portion according to a first embodiment of the present disclosure.

FIG. 9A and FIG. 9B are views illustrating a manual charging portion according to a second embodiment of the present disclosure.

FIG. 10A and FIG. 10B are views illustrating a manual charging portion according to a third embodiment of the present disclosure.

### DETAILED DESCRIPTION

Hereinafter, embodiments of the present disclosure will be described in detail with reference to the attached drawings such that those skilled in the art to which the present disclosure belongs may easily practice the present disclosure. However, the present disclosure may be implemented in various different forms and is not limited to the embodiments described herein. In addition, in order to clearly describe the present disclosure in the drawings, parts that are not related to the description are omitted, and similar components are given similar reference numerals throughout the specification. Also, when description is made with reference to the drawings, even though components are indicated by the same name, reference numbers thereof may change depending on drawings, and the reference numbers of the drawings are described only for the sake of convenience of description, and concepts, features, functions, or effects of the components are not limited by corresponding reference numbers.

In the entire specification of the present disclosure, when a component is described to be “connected” to another component, this includes not only a case where the component is “directly connected” to another component but also a case where the component is “electrically connected” to another component with another element therebetween. In addition, when a portion “includes” a certain component, this does not exclude other components, and means to “include” other components unless otherwise described, and it should be understood that the presence or addition of one or more other features, numbers, steps, operations, components, or combinations thereof is not excluded in advance.

The present disclosure relates to an implantable device for automatic drug administration and may be implanted into the skin of a patient or animal to administer a certain amount of drug at regular intervals. For example, the implantable device may be a small device having a width of 25 mm, a length of 25 mm, and a height of 20 mm and may be made of titanium or a biocompatible three-dimensional (3D) printing material but is not limited thereto.

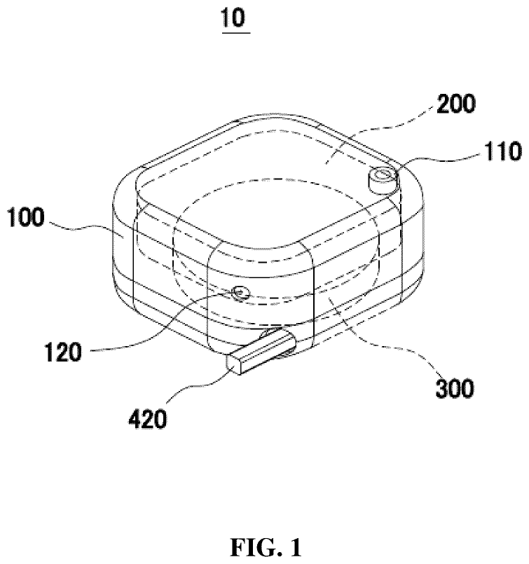

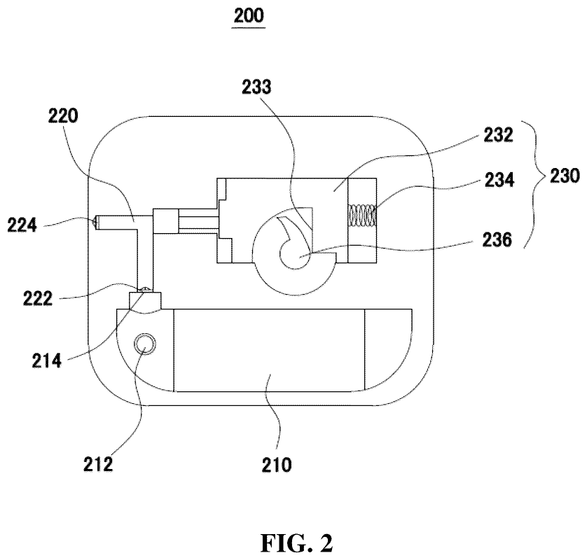

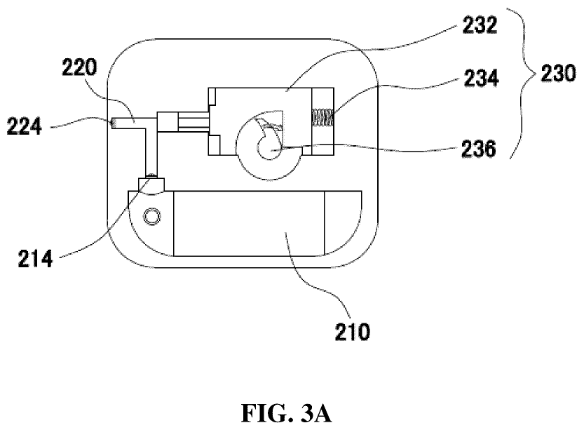

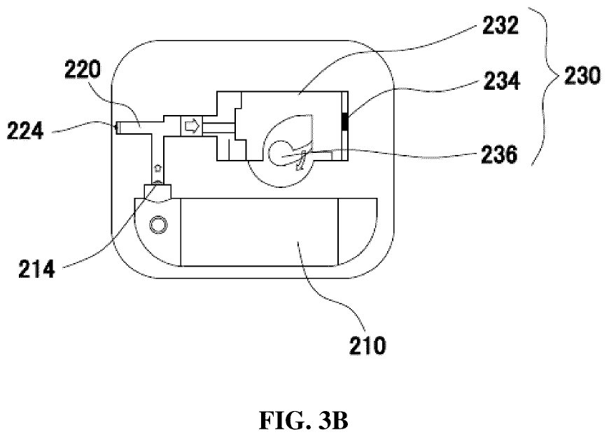

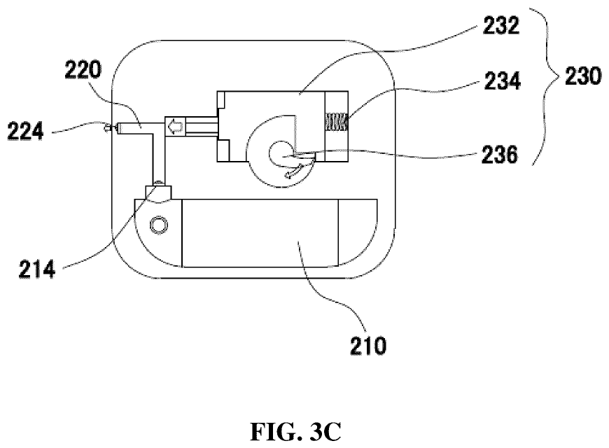

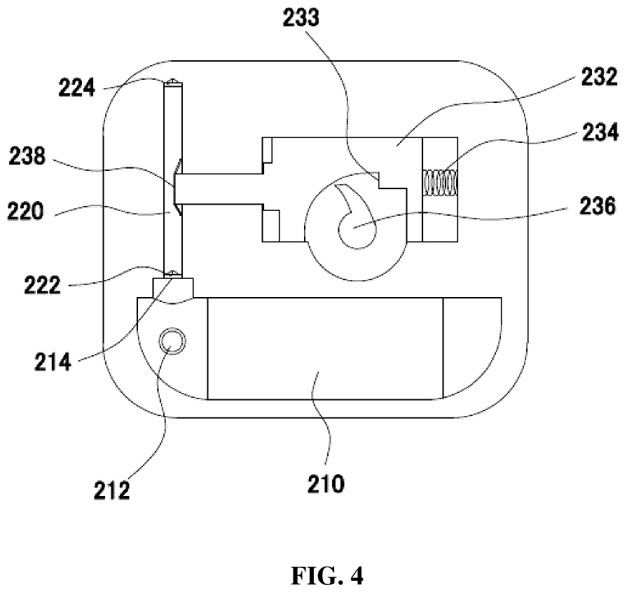

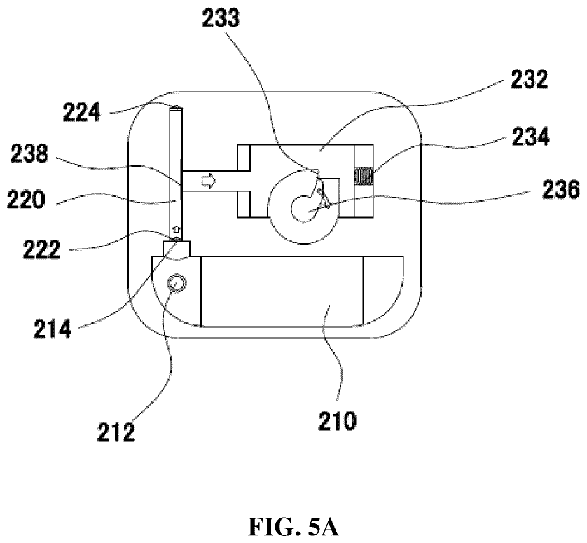

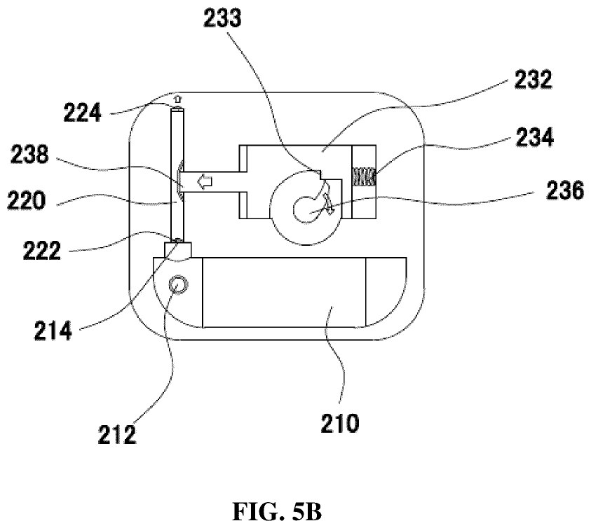

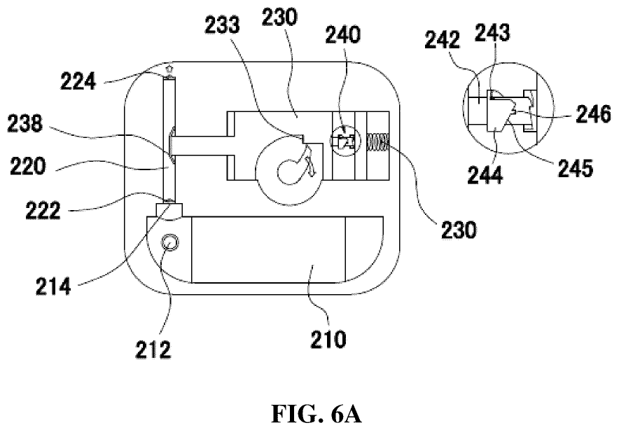

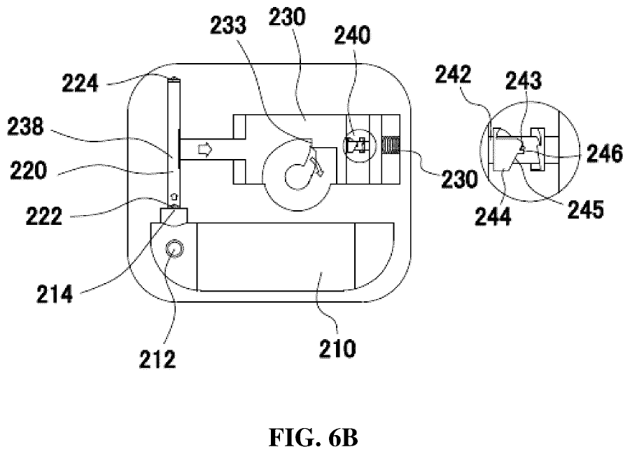

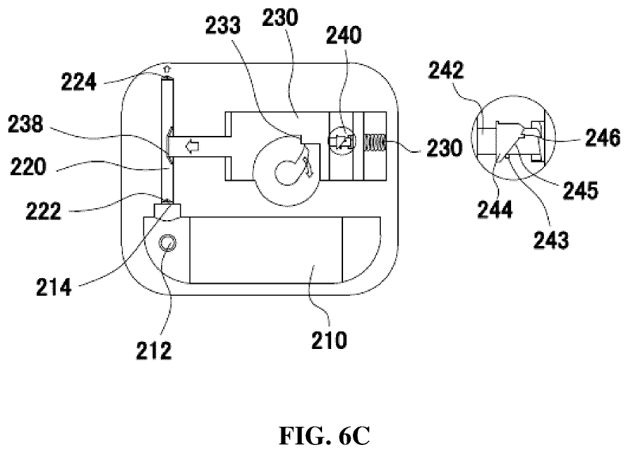

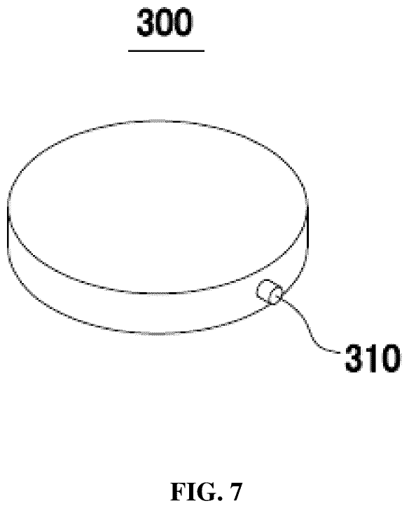

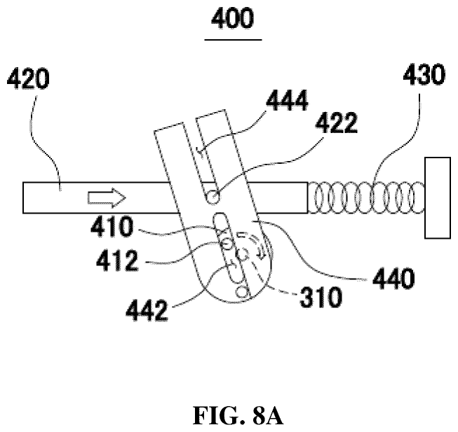

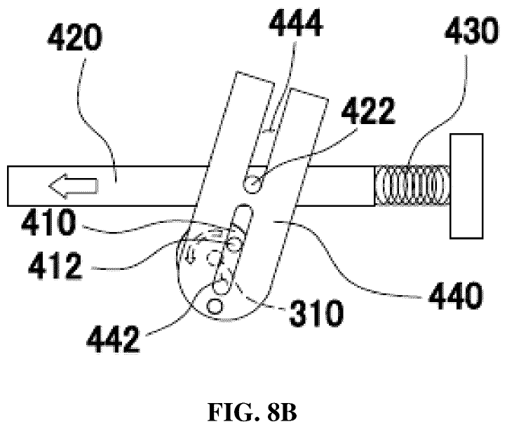

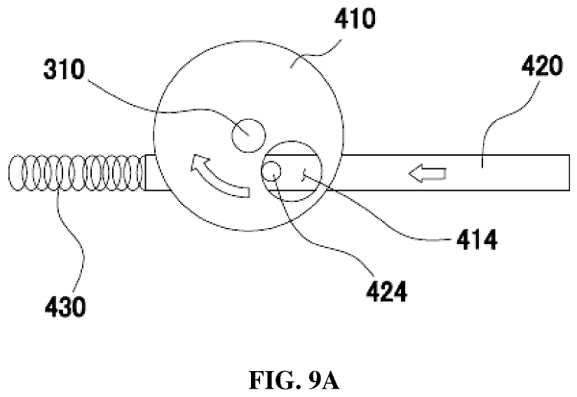

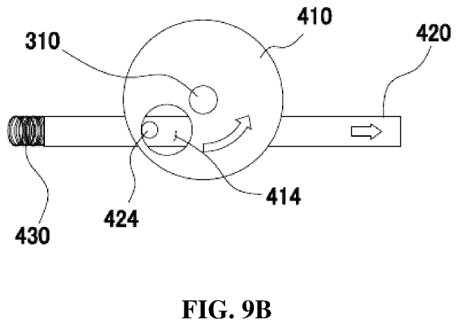

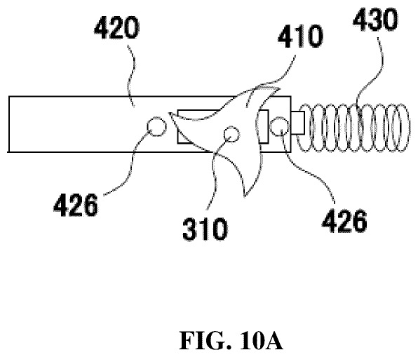

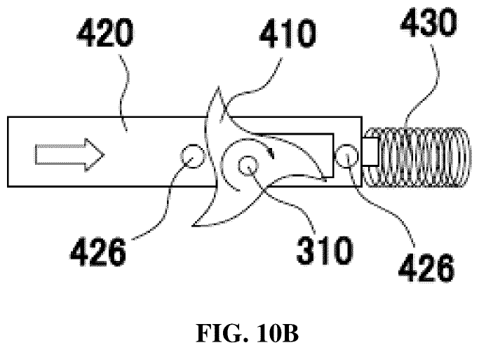

FIG. 1 is a perspective view of an implantable device for automatic drug administration according to an embodiment of the present disclosure, FIG. 2 is a schematic view of a driving unit according to a first embodiment of the present disclosure, FIG. 3A to FIG. 3C are views illustrating an operating mechanism of a driving unit according to the first embodiment of the present disclosure, FIG. 4 is a schematic view of a driving unit according to a second embodiment of the present disclosure, FIG. 5A and FIG. 5B are views illustrating operating mechanisms of a driving unit according to the second embodiment of the present disclosure, FIG. 6A to FIG. 6C are views illustrating a drug administration time controller according to an embodiment of the present disclosure, FIG. 7 is a schematic view of a driving unit according to an embodiment of the present disclosure, FIG. 8A and FIG. 8B are views illustrating a manual charging portion according to a first embodiment of the present disclosure, FIG. 9A and FIG. 9B are views illustrating a manual charging portion according to a second embodiment of the present disclosure, and FIG. 10A and FIG. 10B are views illustrating a manual charging portion according to a third embodiment of the present disclosure.

Hereinafter, an implantable device 10 according to an embodiment of the present disclosure will be briefly described with reference to FIG. 1 and FIG. 2.

The implantable device 10 includes a housing 100, a drug release unit 200, and a driving unit 300.

The housing 100 may include a drug injection hole 110 into which a drug may be injected from the outside and a drug ejection hole 120 through which a drug is ejected to the outside from the drug release unit 200. Also, the drug injection hole 110 may protrude at a position corresponding to a drug inlet 212 of a drug storage 210 to be described below. As the drug injection hole 110 is formed to protrude outward, a user may check a position of the drug injection hole 110 through touch even when the implantable device 10 is implanted into the user's skin. Accordingly, a needle of a syringe filled with a drug may be inserted into the drug injection hole 110 and then the drug may be stored in the drug storage 210 through the drug inlet 212.

The drug ejection unit 200 may eject the drug to the outside. In this case, the implantable device 10 may include a plurality of drug ejection units 200. Accordingly, different drugs may be injected into the plurality of drug ejection units 200 and administered to a user, and the injection amounts and injection cycles of respective drugs may be administered to a user to be different from each other.

Referring to FIG. 2, the drug release unit 200 includes the drug storage 210 that is located inside the housing 100 and stores a drug, a drug chamber 220 receiving a predetermined amount of drug from the drug storage 210 and stores the received drug, and a drug release controller 230 that fills the drug chamber 220 with a drug received from the drug storage 210 and releases the drug filled in the drug chamber 220 to the outside.

Also, the drug chamber 220 may include an inlet valve 222 connecting the drug chamber 220 to the drug storage 210 and an outlet valve 224 connecting the drug chamber 220 to the drug ejection hole 120. In this case, the inlet valve 222 and the outlet valve 224 may be check valves that allow fluid to pass therethrough only in one direction. In other words, the inlet valve 222 may be designed such that, when negative pressure is formed inside the drug chamber 220, the drug stored in the drug storage 210 is supplied to the drug chamber 220 and is not supplied in an opposite direction, that is, the drug stored in the drug chamber 220 is not moved to the drug storage 210. The outlet valve 224 may be designed such that, when positive pressure is formed inside the drug chamber 220, the drug stored in the drug chamber 220 is supplied to the outside through the drug ejection hole 120 and is not supplied in the opposite direction, that is, a foreign material outside the implantable device 10 are prevented from flowing into the drug chamber 220.

The driving unit 300 drives the drug release unit 200 at preset intervals. In this case, the driving unit 300 may be an automatic movement that is charged with energy by a user's movement or an electric movement that is driven by a battery. The automatic movement may be mounted on an automatic wristwatch and operates the wristwatch by storing the kinetic energy generated by a user moving his or her wrist. The electric movement may be mounted on a quartz wristwatch and operates the wristwatch through a battery. In general, a movement mounted on a wristwatch may have small size and includes a plurality of gears that rotate at regular intervals, and a gear portion 236 may be connected to one of the plurality of gears. At this time, the gear portion 236 may drive the drug release controller 230 to drive the drug release unit 200 by rotating at a preset time. A detailed description thereof will be made below.

Hereinafter, the drug release unit 200 according to the first embodiment of the present disclosure will be described with reference to FIG. 2.

The drug release unit 200 includes a plunger 232, a plunger spring 234, and the gear portion 236.

As the plunger 232 moves, positive or negative pressure may be formed in the drug chamber 220. Also, the plunger 232 may have a gear groove 233 into which the gear portion 236 is engaged such that the plunger 232 moves in one direction by rotation of the gear portion 236.

The plunger spring 234 may provide a restoring force to the plunger 232 such that the plunger 232 applies pressure to the drug chamber 220.

The gear portion 236 is connected to the driving unit 300 and may rotate at a preset time. For example, as illustrated in FIG. 2, the gear portion 236 may have a single protrusion, the gear groove 233 may have a fan shape, and accordingly, as the protrusion of the gear portion 236 comes into contact with the gear groove 233 to press the gear groove 233, the plunger 232 may move in the right direction.

Also, as illustrated in FIG. 2, the drug chamber 220 may be bent in an “7” shape but is not limited thereto and may also be formed in a straight-line shape.

Hereinafter, an operating mechanism of the drug release unit 200 according to the first embodiment of the present disclosure will be described with reference to FIGS. 3A to 3C.

As the gear portion 236 rotates clockwise, the protrusion of the gear portion 236 presses the plunger 232 to the right, and accordingly, the plunger 232 may move a predetermined distance to the right. In this case, negative pressure is formed inside the drug chamber 220, and the drug stored in the drug storage 210 may be injected into the drug chamber 220 through the inlet valve 222 (see FIG. 3B).

Thereafter, when a predetermined amount of time passes and the gear portion 236 further rotates clockwise and the protrusion of the gear portion 236 is detached from the gear groove 233 of the plunger 232, the plunger 232 may move to the left by a restoring force of the plunger spring 234. In this case, positive pressure is formed inside the drug chamber 220, and the drug stored inside the drug chamber 220 may be ejected to the outside through the outlet valve 224 (see FIG. 3C).

A drug release unit 200 according to a second embodiment of the present disclosure will be described with reference to FIG. 4.

The drug release unit 200 may further include a film portion 238 that is in contact with an end of the plunger 232 and located in the drug chamber 220. In this case, the drug chamber 220 may be formed in a straight line. Also, in the drug release unit 200, as the plunger 232 moves to the left, the film portion 238 protrude in an inner direction of the drug chamber 220, thereby forming positive pressure inside the drug chamber 220, or as the plunger 232 moves to the right, the film portion 238 may be restored to its original state, thereby forming negative pressure inside the drug chamber 220.

An operating mechanism of the drug release unit 200 according to the second embodiment of the present disclosure will be described in detail with reference to FIG. 4.

As the gear portion 236 rotates clockwise, the protrusion of the gear portion 236 presses the plunger 232 to the right, and accordingly, the plunger 232 may be moved to the right by a predetermined distance, thereby restoring the film portion 238 to its original state. In this case, negative pressure may be formed inside the drug chamber 220, and the drug stored in the drug storage 210 may be moved into the drug chamber 220 through the inlet valve 222 (see FIG. 5A).

Thereafter, when a predetermined amount of time elapses and the gear portion 236 rotates clockwise and the protrusion of the gear portion 236 is separated from the gear groove 233 of the plunger 232, the plunger 232 may move to the left by the restoring force of the plunger spring 234, and the film portion 238 may be protruded in an inner direction of the drug chamber 220. In this case, positive pressure is formed inside the drug chamber 220, and the drug located inside the drug chamber 220 may be ejected to the outside through the outlet valve 224 (see FIG. 5B).

A drug administration time controller 240 according to an embodiment of the present disclosure will be described with reference to FIGS. 6A to 6C.

The drug administration time controller 240 is located between the plunger 232 and the plunger spring 234 and may adjust the time at which the drug is ejected by controlling the movement of the plunger 232.

To this end, the drug administration time controller 240 includes a cylindrical portion 242 having one end connected to the other end of the plunger 232, the other end connected to the plunger spring 234, and an outer circumferential surface on which a latch 243 is formed, and a guide portion 244 having an inclined surface 245 of a spiral shape coming into contact with the latch 243 of the cylindrical portion 242 and including an engagement portion 246 protruding on the inclined surface 245.

Referring to FIG. 6B, even when the protrusion of the gear portion 236 is separated from the gear groove 233 of the plunger 232 because the gear portion 236 rotates clockwise and the protrusion of the gear portion 236 presses the plunger 232 to the right, the plunger 232 may not move to the left by the drug administration time controller 240. In this case, negative pressure is formed inside the drug chamber 220, and the drug stored in the drug storage 210 may be moved into the drug chamber 220 through the inlet valve 222.

In other words, the movement of the latch 243 of the cylindrical portion 242 is restricted by the engagement portion 246 of the guide portion 244, and accordingly, even when the gear portion 236 rotates once, the drug may only be stored inside the drug chamber 220 without being ejected to the outside. Thereafter, as the gear portion 236 rotates once clockwise, the movement restriction of the latch 243 located at the engagement portion 246 of the guide portion 244 is released, and as the latch 243 moves along the inclined surface 245 of the guide portion 244, the plunger 232 may move to the left. In this case, positive pressure is formed inside the drug chamber 220, and the drug located inside the drug chamber 220 may be ejected to the outside through the outlet valve 224 (see FIG. 6C).

That is, when the gear portion 236 rotates twice by the drug administration time controller 240, one time of drug administration may be performed, and assuming that the time for one rotation of the gear portion 236 is 12 hours, one time drug administration may be performed every 24 hours.

Referring to FIG. 7, the driving unit 300 may be a movement that winds a mainspring according to a user's movement and is charged with energy. In this case, the movement has a general configuration used for automatic watches, and accordingly, a detailed description thereof is omitted.

Also, the driving unit 300 may be a movement of which mainspring is wound directly by a user to charge the movement. In other words, a user may periodically wind the mainspring directly to charge the movement before the energy charged in the movement is completely exhausted.

In addition, the driving unit 300 may be a movement of which mainspring is wound only by a user's movement to charge the movement. In other words, when a user's movement is smooth, the mainspring is wound only by the user's movement to charge the mainspring with energy. Also, in addition to charging the mainspring with energy by a user's movement, when the energy of the mainspring is exhausted because the user does not move, the movement may have a structure in which the user directly winds the mainspring to charge the mainspring with energy.

In general, the movement has a protrusion portion 310 that protrudes from one side thereof, and when the energy is exhausted or insufficient because the watch is not used, the protrusion portion 310 may be rotated to charge the movement. The implantable device 10 may further include a manual charging portion 400 that may manually charge the movement by rotating the protrusion portion 310 of the movement when the movement is discharged because a user does not move enough.

Hereinafter, the manual charging portion 400 according to various embodiments of the present disclosure will be described with reference to FIG. 8A to FIG. 10B.

The manual charging portion 400 may include a charging gear 410 connected to the protrusion portion 310 of an automatic movement, a clicker charger 420 having one end protruding out of a housing 100 to rotate the charging gear 410, and a spring portion 430 located at the other end of the clicker charger 420. A user may detect a position of the clicker charger 420 protruding out of the housing 100 by touch, and presses the clicker charger 420, thereby rotating the protrusion portion 310 to charge the automatic movement.

Referring to FIGS. 8A and 8B, the charging gear 410 includes a first protrusion portion 412 having a disc shape, separated from the center of the charging gear 410 by a predetermined distance, and protruding from one side surface of the first protrusion portion 412, and the clicker charger 420 may include a second protrusion portion 422 located at one end of the clicker charger 420 and protruding outward.

Also, the manual charging portion 400 may further include a guide puller 440 rotatably coupled to the housing 100 and having a first insertion groove 442 into which the first protrusion portion 412 is inserted and which is elongated in a vertical direction, and a second insertion groove 444 into which the second protrusion 422 is inserted and which is elongated in the vertical direction.

As illustrated in FIGS. 8A and 8B, when the clicker charger 420 is pressed in the right direction, the guide puller 440 rotates clockwise, and the charging gear 410 connected to the guide puller 440 may also rotate clockwise. Thereafter, when the clicker charger 420 moves to the left by the restoring force of the spring portion 430, the guide puller 440 rotates counterclockwise, and the charging gear 410 connected to the guide puller 440 may also rotate counterclockwise. In this case, the automatic movement may be charged as the protrusion portion 310 connected to the charging gear 410 rotates.

Referring to FIGS. 9A and 9B, the clicker charger 420 may include a third protrusion portion 424 that is located at one end of the clicker charger 420 and protrudes outward, and the charging gear 410 may include a circular hole 414 which is at a predetermined distance from the center of charging gear 410 and is larger than the second protrusion portion 422 and into which the third protrusion portion 424 is inserted.

As illustrated in FIGS. 9A and 9B, the charging gear 410 may rotate counterclockwise as the clicker charger 420 is pressed to the left. Thereafter, as the clicker charger 420 moves to the right by the restoring force of the spring portion 430, the charging gear 410 may rotate clockwise. In this case, as the protrusion portion 310 connected to the charging gear 410 rotates, the automatic movement may be charged.

Referring to FIGS. 10A and 10B, the clicker charger 420 may further include fourth protrusion portions 426 respectively on the left and right sides of the charging gear 410, and the charging gear 410 may rotate as the fourth protrusion portions 426 press the charging gear 410. In this case, the charging gear 410 may have a plurality of protrusions each having one side protruding to be curved and the other side that is recessed and curved.

As illustrated in FIGS. 10A and 10B, as the clicker charger 420 is pressed to the right, the fourth protrusion portion portions 426 on the left side press the protrusion of the charging gear 410, and accordingly, the charging gear 410 may rotate clockwise. Thereafter, as the clicker charger 420 moves to the left by the restoring force of the spring portion 430, the fourth protrusion portions 426 on the right side press the protrusion of the charging gear 410, and accordingly, the charging gear 410 may rotate clockwise. In this case, as the protrusion portion 310 connected to the charging gear 410 rotates, the automatic movement may be charged.

The above description of the present disclosure is intended to be illustrative, and those skilled in the art will appreciate that the present disclosure may be readily modified in other specific forms without changing the technical idea or essential characteristics of the present disclosure. Therefore, the embodiments described above should be understood as illustrative in all respects and not limiting. For example, each component described in a single type may be implemented in a distributed manner, and likewise, components described in a distributed manner may be implemented in a combined form.

The scope of the present application is indicated by the claims described below rather than the detailed description above, and all changes or modified forms derived from the meaning, scope of the claims, and their equivalent concepts should be interpreted as being included in the scope of the present application.

What is claimed is:

1. An implantable device for automatic drug administration, the implantable device comprising:
a housing having a predetermined shape;
a drug release unit configured to eject a drug to an outside; and
a driving unit configured to drive the drug release unit at preset intervals,
wherein the drug release unit comprises a drug storage located inside the housing and storing a drug, a drug chamber receiving a predetermined amount of drug from the drug storage and storing the received drug, and a drug release controller configured to fill the drug chamber with a drug received the drug storage and release the drug filled in the drug chamber to the outside, and
the drug release controller comprises a plunger configured to form one of positive pressure and negative pressure in the drug chamber as the plunger moves, a plunger spring configured to apply a restoring force to a position where the plunger applies presses the drug chamber, and a gear portion connected to the driving unit and configured to rotate at a preset time to drive the drug release unit.

2. The implantable device of claim 1, wherein
the plunger has a gear groove with which the gear portion is engaged such that the plunger moves in one direction by rotation of the gear portion.

3. The implantable device of claim 1, further comprising:
a plurality of drug release units.

4. The implantable device of claim 1, wherein
the drug chamber comprises an inlet valve connecting the drug storage to the drug chamber, and an outlet valve connecting the drug chamber to a drug ejection hole.

5. The implantable device of claim 1, wherein
the driving unit is a movement that is charged with energy as a user directly winds a mainspring, or a movement that is charged with energy as the mainspring is wound by the user's movement.

6. The implantable device of claim 1, wherein
the driving unit is a movement capable of being charged with energy by a user's movement and by the user's action of directly winding a mainspring.

7. The implantable device of claim 1, wherein
the driving unit is an electric movement driven by a battery.

8. The implantable device of claim 1, wherein
the drug release unit further comprises a film portion that is in contact with an end of the plunger and is located in the drug chamber.

9. The implantable device of claim 1, further comprising:
a drug administration time controller located between the plunger and the plunger spring and configured to control a movement of the plunger to control a time at which the drug is ejected,
wherein the drug administration time controller comprises a cylindrical portion having one end connected to another end of the plunger, another end connected to the plunger spring, and an outer circumferential surface on which a latch is formed, and a guide portion having an inclined surface of a spiral shape coming into contact with the latch of the cylindrical portion and including an engagement portion protruding on the inclined surface.

10. The implantable device of claim 1, wherein
the drug storage comprises a drug injection hole into which the drug is introduced from the outside and a drug transfer hole through which the drug in the drug storage is transferred to the drug chamber.

11. The implantable device of claim 10, wherein the housing comprises:
a drug injection hole protruding out of a portion corresponding to a drug inlet; and
a drug ejection hole through which the drug is ejected to the outside from the drug release unit.

12. The implantable device of claim 5, further comprising:
a manual charging portion configured to charge the movement,
wherein the manual charging portion comprises a charging gear connected to a protrusion portion of the movement, a clicker charger having one end protruding out of the housing to rotate the charging gear, and a spring portion located at another end of the clicker charger, and
as the clicker charger is pressed, the protrusion portion rotates to charge the movement.

13. The implantable device of claim 12, wherein
the charging gear comprises a first protrusion portion having a disc shape, separated from the center of the charging gear by a predetermined distance, and protruding from one side surface of the first protrusion portion,
the clicker charger comprises a second protrusion portion 422 located at one end of the clicker charger 420 and protruding outward, and
the manual charging portion further comprises a guide pulling portion rotatably coupled to the housing and having a first insertion groove into which the first protrusion portion is inserted and which is elongated in a vertical direction, and a second insertion groove into which the second protrusion is inserted and which is elongated in the vertical direction.

14. The implantable device of claim 12, wherein
the clicker charger comprises a third protrusion portion located at one end of the clicker charger and protruding outward, and
the charging gear includes a circular hole which is at a predetermined distance from a center of the charging gear and is larger than the second protrusion portion and into which the third protrusion portion is inserted.

15. The implantable device of claim 12, wherein
the clicker charger further comprises fourth protrusion portions located respectively on left and right sides of the charging gear, and
the charging gear rotates as the fourth protrusion portions press the charging gear.
